(19) United States
(12) Patent Application Publication
(10) Pub. No.: US 2024/0209464 A1
(43) Pub. Date: Jun. 27, 2024

(54) Pathogen Detection From Urine Analyte in All Gender Patients

(71) Applicant: Brian Joseph Hajjar, Palisades Park, NJ (US)

(72) Inventor: Brian Joseph Hajjar, Palisades Park, NJ (US)

(21) Appl. No.: 18/393,986

(22) Filed: Dec. 22, 2023

Related U.S. Application Data
(60) Provisional application No. 63/475,895, filed on Dec. 27, 2022.

(51) Int. Cl. C12Q 1/70; C12Q 1/689

(52) U.S. Cl. CPC C12Q 1/707; C12Q 1/689; C12Q 1/703; C12Q 1/705

(57) ABSTRACT

The present invention allows for the detection of pathogens in a urine specimen, such as bacteria and viruses, including HCV, STDs, and HIV in a urine analyte in patients. The invention accomplishes the detection with methods that employ amplification of DNA from cells in the urine specimen, for example, by using a PCR-based assay. The method can detect one or more high-risk forms of HCV, STDs, and HIV 1 and 2. The method is useful for all genders, including cisgender, transgender and nonbinary patients and can achieve high analytical sensitivity.

The present application claims the benefit of priority under 35 U.S.C. § 119(e) of Provisional Application No. 63/475,895, filed on Dec. 27, 2022, the entire disclosure which is hereby incorporated by reference.

### BACKGROUND OF THE INVENTION

### Field of the Invention

The present invention is directed to the detection of pathogens from urine samples, and in particular Hepatitis C virus (HCV), Sexually Transmitted Diseases (STDs), Herpes Simplex Virus (HSV), syphilis and Human Immunodeficiency virus (HIV) from urine samples.

### DESCRIPTION OF RELATED ART

HCV, STDs and HIV can be detected in urine using assays but deficiencies in the established detection methods render such methods not useful. The specificity in urine and test performance characteristics are not acceptable for regulatory approval or widespread screening.

The implementation of low-cost screening and treatment campaigns for HCV, STDs and HIV have been limited by lack of availability of point-of-care tests. Polymerase chain reaction (PCR) tests rely on blood analyte for screening and are not conducive to at home testing. Previous PCR assays have lacked sensitivity and specificity. At least one problem has been the presence of soluble PCR inhibitors in urine.

PCR urine tests are not available for HCV, STDs, HSV, syphilis and HIV. Additionally, some urine tests are limited to a particular gender. There is no all-gender PCR urine test for HCV, STDs, HSV, syphilis and HIV. There are also no PCR urine tests capable of detecting multiple pathogens from a single urine specimen.

Transgender and non-binary people who are gay, bisexual or otherwise and who may have sex with men are undertested for HCV, STDs and HIV but have a similar infection risk as cisgender patients. They may face complex transintersectional barriers to testing such as non-inclusive clinic environments, lack of discretion in services, and lack of provider knowledge and competency. Home testing kits using a urine specimen would reduce the stigma and barriers these patient groups face regarding testing and may facilitate better treatment options and outcomes.

There is a need for development of robust tests via urine to detect HCV, STDs, HSV, syphilis and HIV in all-gender patients before they develop complicated disease or unknowingly transmit these viral and microbiological pathogens to their partners during sexual intercourse.

Chronic hepatitis C is associated with multiple extrahepatic manifestations that may affect the infected patients. Hepatitis C induced kidney disease is a devastating complication in patients with HCV infection. HCV infection is the leading cause of liver transplantation in the US and is a risk factor for liver cancer. HCV and other forms of viral hepatitis are on the rise internationally and is associated with sexual contact. Sexual contact has emerged as the most frequent transmission route of HCV infection. There is no fast and clinically relevant urine-based test available for patients that employs amplifying the DNA from cells in the urine.

HIV rates rose 16% in 2021 and testing decreased due to the COVID-19 public health emergency. HIV is a virus that attacks the immune system. As an HIV infection progresses, it increases the risk of cancer and life-threatening infections. In the United States (U.S.), HIV is primarily spread through sex or sharing of needles with a person infected with HIV. Since HIV does not always present symptoms, it is possible to have the virus and not be aware of it. About one in eight people in the U.S. don't know that they have HIV, according to HIV.gov. The need for self-testing has never been greater. The Centers for Disease Control and Prevention (CDC) recommends that everyone be tested at least once for HIV between the ages of 13 and 64. For high-risk patients, more regular testing is recommended. At-home HIV testing checks the blood or oral fluid. At home test kits currently use a mouth swab sample for antigens and/or antibodies that are produced in response to the virus. An HIV urine detection test was approved in the 1990s but it is not based on PCR technology or other DNA amplification technology and is rarely used today. The only PCR test for HIV involves blood or plasma samples for assay. There is no urine PCR HIV test available for patients.

STDs have been increasing worldwide since the 1990s and the current epidemic has raised public concerns. At-risk individuals, such as people living with HIV (PLWH) who are men who have sex with men (MSM), are more frequently infected with STDs compared with HIV-negative individuals. STDs include, but are not limited to, trichomoniasis, mycoplasma genitalium, chlamydia, gonorrhea, syphilis and herpes.

A rise in the incidence of gonorrhea and chlamydia has occurred in at-risk populations with evolutional changes of sexual behaviors and introduction of effective antiretroviral therapy. Chlamydia trachomatis (CT) and Neisseria gonorrhoeae (NG) are among the most prevalent sexually transmitted pathogens worldwide. Regular screening is recommended for at-risk sexually active individuals. Concurrent infection with CT and NG in MSM is not uncommon. There are several PCR tests available for at home testing (CT/NG/TV) but they are limited by the requirement for vaginal swab specimen in women. Additionally, currently available urine testing methods may not be as effective as doing similar tests on a vaginal swab. One kit does test urine in males but is for point of care or physician office use but not home use. There is no at-home PCR test kit for testing CT/NG/TV using urine as a specimen so that testing is feasible for all genders, including cisgender, transgender and non-binary persons.

Syphilis is a bacterial disease that surfaces as genital sores but can ultimately lead to severe symptoms and death if left untreated. Congenital syphilis cases, in which infected mothers pass the disease on to their babies, potentially leading to death of the child or health problems such as deafness and blindness, is on the rise. The rates of syphilis have reached the highest in three decades (i.e. 16 per 100,000 people) causing the CDC to call for new prevention and treatment to rebuild, innovate and expand STD prevention in the U.S. Testing currently requires a blood specimen. There are no commercial urine tests for syphilis and no PCR tests available. There is no at-home PCR test kit for testing syphilis using urine as a specimen for all genders, including cisgender, transgender and non-binary persons.

Trichomoniasis, caused by Trichomonas vaginalis (TV), is the most prevalent nonviral sexually transmitted infection in the U.S. Infections often go unnoticed, leading to asymptomatic spread of infection. Infections in women can lead to vaginitis, cervicitis and urethritis and in more severe cases complications such as premature labor, low-birth weight offspring, premature rupture of membranes, pelvic inflammatory disease (PID), tubal infertility, and cervical cancer. Symptoms in men who have sex with women include urethritis, epididymitis, or prostatitis. Diagnostic screening should be performed in women with vaginal discharge, for persons receiving care in high-prevalence settings such as correctional facilities and SDT clinics, and for asymptomatic persons at high risk for infection such as persons with multiple sexual partners, illicit drug use, or a history of STDs. Testing is typically not performed on males. There is no at-home PCR test kit for testing trichomonas using urine as a specimen for all genders, including cisgender, transgender and non-binary persons.

Mycoplasma genitalium (MG) is an often overlooked sexually transmitted infection which can lead to urethritis in men and cervicitis, endometritis, PID, infertility, susceptibility to human immunodeficiency virus (HIV), and adverse birth outcomes in women. Screening is performed in high-risk individuals. Molecular testing with a nucleic acid amplification test is performed using vaginal or ureteral swab specimens in males. There is no at-home PCR test kit for testing MG using urine as a specimen for all genders, including cisgender, transgender and non-binary persons.

Prior to molecular diagnostics, TV/MG infections were detected with culture of vaginal secretions in women and urethral swab, urine sediment, and/or semen in men. Sensitivity was poor and wet preparation of genital secretions has become the more common detection method, although the sensitivity is only 35-80% that of microscopic culture. Sensitivity also declines by up to 20% within 1 hour of specimen collection. There is a need for the facile detection of TV in all genders, including cisgender, transgender, and non-binary persons. There is a need for a quick, inexpensive, and reliable method to test for TV in urine specimens which are either self-collected or clinician collected.

Herpes simplex virus (HSV) type 1 and 2 can lead to both oral and genital herpes. Both oral and genital herpes are mostly asymptomatic or unrecognized but can cause painful blisters or ulcers at the site of infection, ranging from mild to severe. Infection is lifelong, and symptoms can recur over many years. Herpes can be transmitted from mother to child during delivery, causing neonatal herpes. HSV-2 infection increases the risk of acquiring HIV infection by approximately three-fold. Additionally, people with both HIV and HSV-2 infection are more likely to spread HIV to others. HSV-2 infection is among the most common infections in people living with HIV. People with symptoms suggestive of genital herpes should be offered HIV testing. Current testing for herpes uses blood as a specimen. PCR testing also requires blood or a sample of fluid from a sore as a specimen. Only a PCR blood test can distinguish between HSV-1 and HSV-2. There are no commercial urine tests for herpes. There is a need for a urine analyte based PCR test. There is no at-home PCR test kit for testing HSV using urine as a specimen in cisgender, transgender and non-binary persons.

Testing for other STDs has been low because culture methods are time-consuming, expensive and labor intensive procedures which require specific and careful specimen handling and transport conditions for viable organisms to reach the laboratory. Culture is not always feasible, especially where there are long distances between clinical and diagnostic facilities or in resource poor settings. Molecular tests (e.g. PCR) are costly, not widely available and require specimens other than urine. Although more sensitive than cultures, previous attempts to use urine specimens with molecular diagnostics such as PCR testing have not been sensitive or accurate. A validated laboratory-developed (LDT) real-time assay is needed, for example, a PCR-based assay.

Since high-risk patients often have one or more infections concurrently, e.g. HIV and a STD, there is a need to test for coinfections. There is a need for bundled testing of high-risk individuals. There is a need for inexpensive point-of-care or over-the-counter tests to facilitate and increase screening in key populations, including cisgender, transgender and non-binary people.

Improving screening for HCV, STDs and HIV facilitates access to appropriate prevention and treatment. Current guidelines recommend regular screening for chlamydia, gonorrhea, and syphilis in sexually active PLWH who are MSM. Effective and validated screening for sexually transmitted pathogens is needed for appropriate management of infected individuals and their partners, and to prevent further transmission. Urine testing makes it easier for people to undergo STI testing as part of their regular medical care. At home test kits and point of care testing is needed.

### SUMMARY OF THE INVENTION

The present invention relates to methods for determining the presence or absence of a pathogen, such as HCV, STDs, HSV, syphilis and HIV, and in particular nucleic acid molecules of the pathogens, using one or more nucleic acid amplification tests, such as PCR in urine samples. The methods are useful for all genders, including cisgender, transgender and non-binary person.

The present invention relates to an at-home test kit for testing HCV using urine as a specimen in cisgender, transgender and non-binary persons.

The present invention relates to an at-home test kit for testing CT/NG/TV using urine as a specimen.

The present invention relates to an at-home test kit for testing syphilis using urine as a specimen.

The present invention relates to an at-home test kit for testing trichomonas using urine as a specimen.

The present invention relates to an at-home test kit for testing MG using urine as a specimen.

The present invention relates to an at-home test kit for testing HSV using urine as a specimen.

The present invention relates to an at-home test kit for testing HIV using urine as a specimen.

The present invention relates to an at-home test kit for testing multiple pathogens using a single urine specimen.

The present invention relates to development and validation of laboratory-developed tests and will allow clinicians to easily detect these pathogens in their patients.

The present invention relates to development and validation of laboratory-developed tests for detection of pathogens, such as HCV, STDs and HIV using self-collection urine analyte methods.

The present invention is directed to a method of detection of multiple pathogens, HCV, HIV, STDs, from a single urine specimen, permitting detection of all these pathogens simultaneously (i.e. test bundling).

The present invention presents a clinically validated method, such as a clinically validated PCR-based method, for detection of one or more of pathogens including HCV, STDs and HIV alone or in combination in a single urine specimen.

An object of the present invention is to provide a PCR-based method using a urine analyte which can detect one or more high-risk forms of HCV subtypes, STDs, and HIV in the Cobas® 6800/8800 assay with high analytical sensitivity.

An object of the present invention is to provide a gender neutral PCR-based method which can detect one or more high-risk forms of HCV, STDs and HIV and other subtypes in the Cobas® 6800/8800 assay in all genders, including cisgender, transgender and non-binary patients.

The present invention provides an improved method for pathogen detection, such as HCV, STD, HSV, syphilis and HIV detection, by the PCR-based testing of urine samples.

An object of the present invention is to perform tests for HCV, STD, HSV, syphilis and HIV either separately or bundled in combination using a single urine sample.

An object of the present invention is to test for chlamydia and/or gonorrhea among PLWH, especially those co-infected with HCV and syphilis.

An object of the present invention is to provide a PCR-based method which can detect HCV, STD, HSV, syphilis and HIV in the Cobas® 6800/8800 assay in patients with high analytical sensitivity.

It is an object of the present invention to provide clinical validation for the Cobas 6800/8800 HCV, STD, HSV, syphilis and HIV tests for all-gender patients, including cisgender, transgender and nonbinary people.

It is an object of the present invention to provide a semi-quantitative test for HCV and HIV viral load.

According to the present invention as described above, since it is possible to obtain a highly specific and reproducible detection of HCV, STDs, HSV, syphilis and HIV in asymptomatic patients, the test may confidently be adopted into clinical practice.

Another advantage is that it is possible to automatically perform the identification of HCV, STDs, HSV, syphilis and HIV in urine analyte.

Another advantage is a non-invasive, easily accessible, screening test capable of detecting subclinical HCV, STDs, HSV, syphilis and HIV infections in patients or clinicians to perform at the point of care without great cost or discomfort to patients. The tests can be performed more frequently than current screening methods and at home.

It is an object of the present invention to provide in vitro detection of HCV, STDs, and HIV in clinician or patient-collected urine specimens.

It is an object of the present invention to provide HCV, STD, HSV, syphilis and/or HIV testing in cis- gender, transgender and non-binary people useful for routine screening. This, in turn, will provide the opportunity to develop pathways for the management of positive test results including instituting appropriate medical therapy and reducing health care costs.

It is an object of the embodiments of the present invention to provide a non-invasive home-based urine collection method which allows for patients to self-sample and return the sample to a laboratory for pathogen testing, including HCV, STD and HIV testing, by mail.

It is an object of the present invention to provide patients with a sensitive and specific HCV, STD, HSV, syphilis and HIV self-sampling urine collection screening option.

It is an object of the present invention to provide PCR identification of HCV, STDs, HSV, syphilis and HIV which correlates with clinical significance.

In some embodiments the method employs a platform, such as a PCR technology platform, which is integrated with data management systems, locally, regionally and nationally to allow for effective epidemiological surveillance and control of disease outbreaks.

Other nucleic acid amplification technologies that may be employed in the present invention are well known to those skilled in the art and may include, inter alia, loop mediated isothermal amplification, nucleic acid sequence based amplification, strand displacement amplification, and multiple displacement amplification. In a preferred embodiment, the nucleic acid amplification assay of the present invention is PCR.

In some embodiments, the patient may be seen in an acute care setting such as an emergency department or hospital.

Another embodiment of the invention comprises diagnostic kits for pathogen detection, such as HCV, STD, HSV, syphilis and HIV detection, PCR sequencing and analysis according to the methods disclosed herein. In some embodiments, the kit includes instructions relating to its use.

In some embodiments, the kits include a urine specimen collection device or container and/or reagents.

In one embodiment of the present invention, a kit is used for diagnosing or confirming with connectivity through the Cloud, an App, a computer, artificial intelligence (AI) or through other electronic means.

In some embodiments, nucleic acid amplification and detection methods useful for the diagnosis and management of a variety of HCV, STD, HIV strains considered to be high-risks are used.

### BRIEF DESCRIPTION OF THE DRAWINGS

FIG. 1 shows the HCV LOD parameters. Based on LOD data low copy number of 25 copies/ml, mid 500 copies/ml and high 1000 copies/ml of the determined LOD and 10 negative samples.

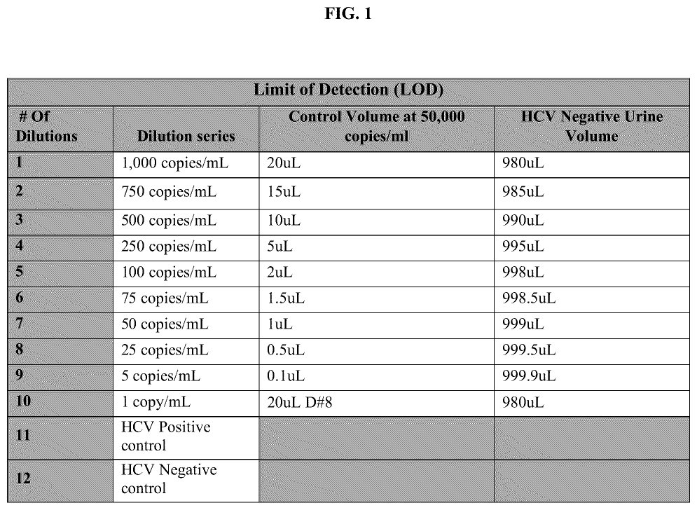

FIG. 2 illustrates the HCV LOD data according to an exemplary embodiment of the invention.

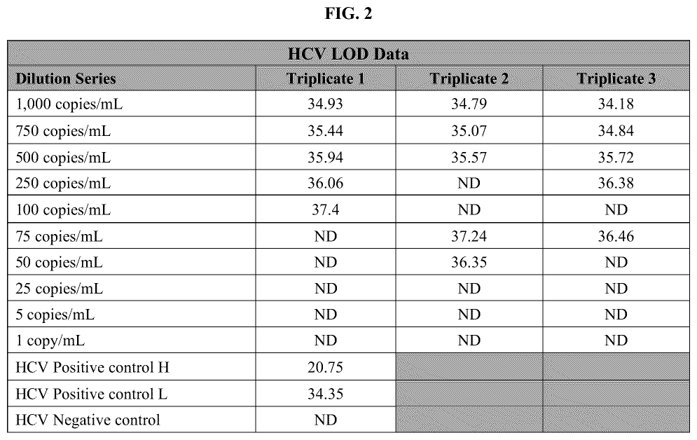

FIG. 3 is a graph showing a summary of HCV LOD results according to an exemplary embodiment of the invention.

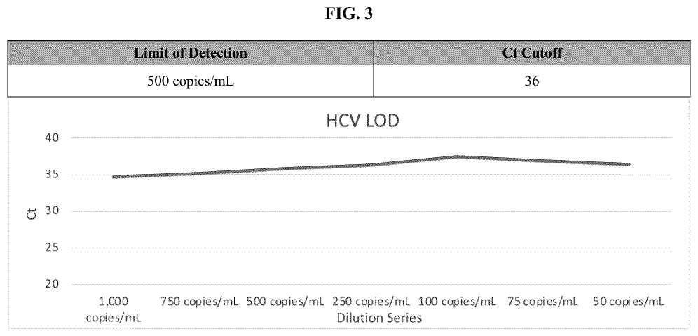

FIG. 4 presents accuracy and reproducibility of a HCV assay according to an exemplary embodiment of the invention.

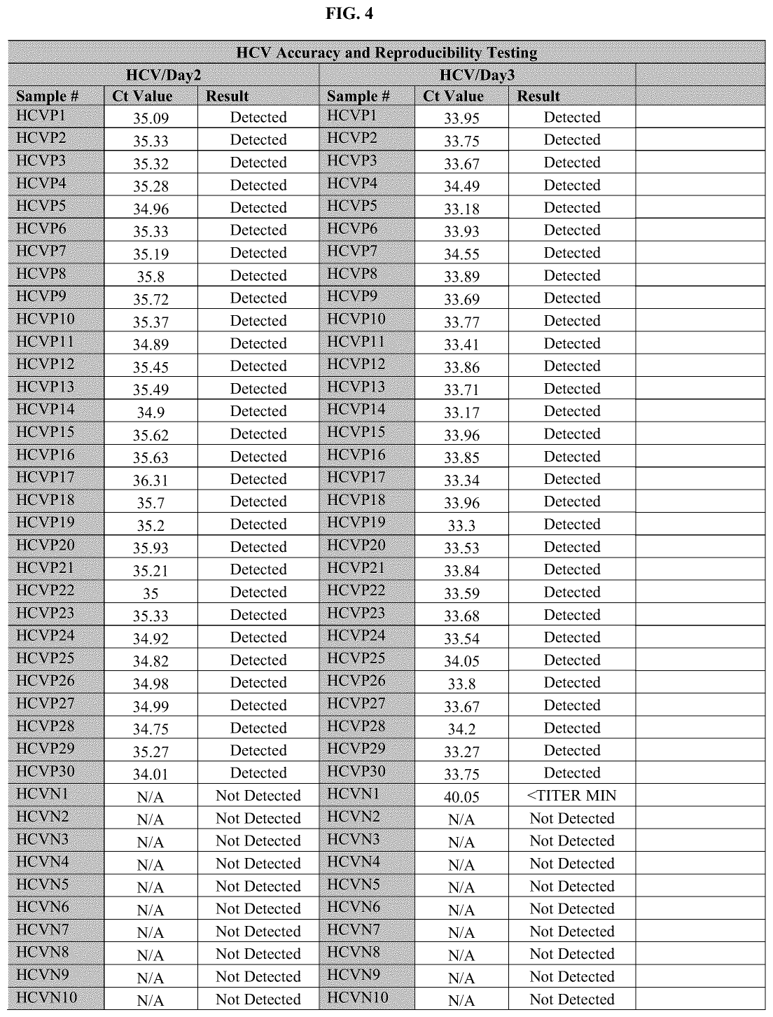

FIG. 5 illustrates a summary of the clinical validation of a HCV test according to an exemplary embodiment of the invention.

FIG. 6 shows stability data for the HCV test according to an exemplary embodiment of the invention

FIG. 7 is a graph of the stability data of FIG. 6

FIG. 8 shows HIV validity data according to an exemplary embodiment of the invention.

FIG. 9 illustrates the HIV-1 LOD data according to an exemplary embodiment of the invention.

FIG. 10 illustrates the HIV-2 LOD data according to an exemplary embodiment of the invention.

FIG. 11 is a graph showing the HIV-1 limit of detection according to an exemplary embodiment of the invention.

FIG. 12 is a graph showing the HIV-2 limit of detection according to an exemplary embodiment of the invention.

FIG. 13 is a summary of the HIV-1 and HIV-2 accuracy and reproducibility testing according to an exemplary embodiment of the invention.

FIG. 14 provides a summary of clinical validation testing for HIV-1 and HIV-2 according to an exemplary embodiment of the invention.

### DETAILED DESCRIPTION

The disclosure provides methods for the preparation and detection of pathogens, such as HCV, STDs, HSV, syphilis and HIV, in urine samples provided by patients or clinicians. No currently validated method to test urine analyte for pathogens such as HCV, HSV, syphilis and HIV in patients using a DNA amplification method, such as PCR, exists. The Cobas® HCV and HIV tests specify that it is for use with a blood test. The disclosure provides for a test that is gender neutral, with no age limitations, which is urine-based instead of traditional blood based.

Transgender and non-binary populations face a unique set of health challenges, including barriers to achieving optimal preventive/screening for HCV, STDs and HIV and lack of access to health care. In one aspect of the present invention, the PCR-based urine assay for HCV, STDs, HSV, syphilis and HIV is useful for facilitating detection and removing barriers to testing in these patient populations.

Aspects of the disclosure described herein may aid HCV, STD, HSV, syphilis and HIV testing methods in patients and lead to improved patient outcomes. The availability of a HCV, STD, HSV, syphilis and HIV urine test in a few hours or less, as opposed to a few days, could potentially decrease transmissibility.

The present invention overcomes disadvantages associated with current strategies and provides tools, and other methods to facilitate and simplify PCR urine testing for HCV, STD, HSV, syphilis and HIV, facilitating testing for all genders, and in particular in underserviced genders such as cis gender, transgender and non-binary patients.

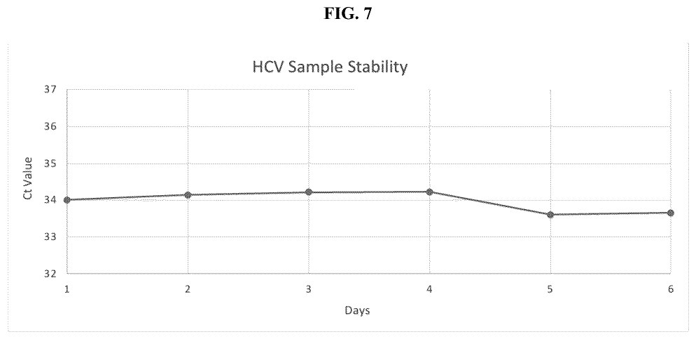

Pathogen nucleic acid amplification, such as PCR, is a more sensitive method for HIV detection in that it reduces the window period of fourth generation immunoassay tests (i.e. the several week window period during acute infection before the onset of a detectable antibody response). Unlike the existing PCR test which relies on ETDA plasma or serum, the present invention uses a urine sample. FIG. 7 shows the data from the assay.

Certain embodiments of the present invention include, but are not limited to, a method for testing for the presence of a pathogen in a patient sample comprising:
1) providing a urine specimen from a patient
2) extracting DNA from cells or pathogens in the urine specimen to obtain extracted DNA,
3) amplifying the extracted DNA, and

4) testing the amplified DNA for the presence of DNA from a pathogen.

The patient being tested by the method may be cisgender, transgender or nonbinary and may benefit from the testing method because the method may be more accessible to the patient.

The urine sample of the method is preferably an unaltered urine specimen that may be easily obtained by a patient at home or in the doctor's office, either assisted or unassisted.

The method preferably employs an automated system capable of performing steps 1-4 without the use of manual manipulation.

The DNA from the cells of the urine specimen may be extracted according to any means known by a skilled artisan and may also be amplified by any means, including PCR amplification.

The method is capable of testing for one pathogen or multiple pathogens at the same time from the urine specimen. Nonlimiting examples of pathogens include human immunodeficiency virus (HIV), hepatitis C virus (HCV), herpes simplex virus (HSV), syphilis, Chlamydia trachomatis (CT) and Neisseria gonorrhoeae (NG), Mycoplasma genitalium (MG) and Trichomonas vaginalis (TV).

The extracting, amplifying and testing of the method according to the invention may be performed by, for example, a Cobas® system as further discussed herein.

The method preferably further comprises determining whether the patient is positive or negative for the pathogen and may further comprises generating a report indicating whether the patient is positive for the pathogen which may be reviewed by a doctor or the patient.

Certain embodiments of the present invention include, but are not limited to, a method for testing for the presence of HCV, HIV, HSV and/or syphilis in a patient sample comprising:
1) providing a urine specimen from a patient
2) extracting DNA from cells or pathogens in the urine specimen to obtain extracted DNA,
3) amplifying the extracted DNA, and
4) testing the amplified DNA for the presence of DNA from a HCV, HIV, HSV and/or syphilis.

In a preferred embodiment, the method may be for testing for the presence of HCV in a patient sample comprising
1) Providing a urine specimen from a patient
2) extracting DNA from pathogens or cells in the urine specimen (extraction process)
3) subjecting the DNA in the sample to PCR amplification process(Cycle Thresholds)
4) testing the amplified DNA for the presence HCV
5) optionally generating a report for a physician/patient for review

Certain embodiments of the present invention include, but are not limited to, the method for testing for HIV in a patient sample comprising:
1) Providing a urine specimen from a patient
2) extracting DNA from pathogens or cells in the specimen (extraction process)
3) subjecting the DNA to PCR amplification process (Cycle Thresholds)
4) testing the amplified DNA for the presence of HIV, including HIV1/HIV2
5) optionally generating a report for physician/patient review

Certain embodiments of the present invention include, but are not limited to, the method for testing for the presence of another STD, such as CT, NG or TV in a patient sample comprising:
1) Providing a urine specimen from a patient
2) extracting DNA from pathogens or cells in the specimen (extraction process)
3) subjecting the extracted DNA to PCR amplification process (Cycle Thresholds)
4) testing the amplified DNA for the presence of STD, such as CT, NG or TV
5) optionally generating a report for physician/patient review

Certain embodiments of the present invention include, but are not limited to, the method of the invention as follows:
1) Providing an unaltered urine specimen from a patient
2) extracting DNA from cells of the specimen (extraction process)
3) amplifying the DNA by PCR amplification process (Cycle Thresholds)
4) testing the amplified DNA for the presence of MR or HSV
5) optionally generating a report for physician/patient review

The specimen in step one is preferably a first void urine sample. However, the test is valid without the need for first void urines. Use of first void urine may reduce rates of invalid calls/results. In a preferred embodiment, the urine sample is collected via Specimen Container McKesson 120 mL. The sample is preferably stored at 2-30° C. for up to 7 days prior to performing the Cobas® HCV test. The sample is not preserved or centrifuged into a pellet. 1 mL of the sample is loaded on Cobas for amplification.

In one embodiment of the invention, Cobas 6800, which is approved by FDA for HCV and HIV detection is used to extract, amplify and test the DNA for the presence of a pathogen. However, it is understood that the assay may also be performed on any apparatus capable of detecting pathogens, such as the Cobas® 8800 system. In accordance with the invention, any system that can detect the presence of a pathogen from a urine sample may be used. Such systems include Hologic Panther Manual workflow. In addition, the systems may include QuantStudio 12K Flex System and Sentosa SA201 Real-Time PCR Instrument.

The success of screening for HCV, HSV, STDs and HIV depends on the reliability and sensitivity of the test used. The Cobas tests are based on a fully automated sample preparation (nucleic acid extraction and purification) followed by PCR amplification and detection. The Cobas tests for use on the 6800 system has not been validated using urine analyte. The Cobas tests processed on the Cobas 6800 system has automated sample preparation and high-throughput (over 1,000 samples in 24 hours).

The Cobas® 6800/8800 Systems consists of the sample supply module, the transfer module, the processing module, and the analytic module. Automated data management is performed by the Cobas® 6800/8800 software which assigns test results for all tests as positive, negative, or invalid. Results can be reviewed directly on the system screen and exported or printed as a report. The assay has a total duration of 3 hours. Tests for HCV serotypes 1-6 and various subtypes, 2a-c, 3a; HIV types 1 and 2 are possible (HIV-1, HIV-2) as wells as subgroups M, N, O and P; and herpes simplex virus type 1 (HSV-1) and herpes simplex virus type 2 (HSV-2).

The terms Cobas 6800 and 8800 are used interchangeably without limitation. Other improved assays may be employed.

In one embodiment, PCR amplification and detection occur in a single tube, where probes with different reporter dyes track the different targets in the reaction with B-globin as the control for extraction and amplification adequacy.

In an embodiment, the nucleic acid (DNA) from patient samples may be extracted. The urine sample may be treated to release the nucleic acid by addition of proteinase and lysis reagent The released nucleic acid may be subject to binding to silica surfaces of added magnetic glass particles.

Unbound substances and impurities, such as denatured protein, cellular debris and potential PCR inhibitors may be removed with subsequent wash steps.

Purified nucleic acid may then be eluted from the magnetic glass particles with elution buffer at elevated temperature.

External controls (positive and negative) may be processed in the same way with, for example, each Cobas® HCV run. HCV positive ingredients include: Tris buffer, <0.05% EDTA, <0.1% Sodium azide, <0.01% Non-infectious plasmid DNA (microbial) containing HCV 1, HCV 2 and HCV 3 sequences, <0.01% Non-infectious plasmid DNA (microbial) containing β-globin sequences, <0.002% Poly rA RNA (synthetic).

### Accuracy for Negative Controls

The expected outcomes should be that all negative controls are all negative. Therefore, in a preferred embodiment, the accuracy should be at 100% for the negative controls.

### Negative Control Samples

1. ENTC=ENTC Sample with transport medium only.
2. DNTC=NTC Sample with nuclease free water only.

### Example 2 Accuracy for Positive Controls

All known positive control samples should be positive to reach 100% accuracy. The preferred acceptable range is 95-100%. The accuracy should be at 100% for the positive controls.

A thermostable DNA polymerase enzyme is preferably used for PCR amplification. The HCV and β-globin sequences are used as internal controls to determine the quality of DNA samples and presence of potential inhibitory substances. The control tells us if the DNA extraction was successful and if the PCR works. For a sample to be valid this control gene needs to be amplified simultaneously utilizing a universal PCR amplification profile with predefined temperature steps and number of cycles.

The master mix preferably includes deoxy uridine triphosphate (dUTP), instead of deoxythymidine triphosphate (dTTP), which is incorporated into the newly synthesized DNA (amplicon). Any contaminating amplicon from previous PCR runs are eliminated preferably by the AmpErase® enzyme (uracil N-glycosylase (UNG), which is included in the PCR master mix, during the first thermal cycling step. However, newly formed amplicon are not eliminated since the AmpErase® enzyme is inactivated once exposed to temperatures above 55° C. Cobas® HCV master mix contains detection probes specific for twelve high-risk HCV target sequences. These are primers for detecting HCV serotypes. One detection probe specific for the HCV 1 target sequence, one detection probe specific for the HCV 2 target sequence and one for β-globin. When not bound to the target sequence, the fluorescent signal of the intact probes is suppressed by a quencher dye. During the PCR amplification step, hybridization of the probes to the specific single-stranded DNA template results in cleavage of the probe by the 5′ to 3′ exonuclease activity of the DNA polymerase resulting in separation of the reporter and quencher dyes and the generation of a fluorescent signal. With each PCR cycle, increasing amounts of cleaved probes are generated and the cumulative signal of the reporter dye increases concomitantly.

Real-time detection and discrimination of PCR products (i.e. amplified DNA amplicon or resulting DNA sequence from PCR reaction) is preferably accomplished by measuring the fluorescence of the released reporter dyes for the HCV targets and β-globin, respectively. The Cobas® 6800 System consists of the sample supply module, the transfer module, the processing module, and the analytic module. Automated data management is performed by the Cobas® 6800 software which assigns test results for all tests as positive, negative, or invalid. Positive and negative refer to the determination of the Cobas 6800 instrument.

In real-time PCR, the DNA is amplified and simultaneously quantified. Variations of the general PCR method are known in the art. Results can be reviewed directly on the system screen and exported or printed as a report. Other variations of the described testing methods are contemplated in accordance with the invention.

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example.

As used herein, the term “PCR” (polymerase Chain reaction) describes a method for increasing the concentration of a segment of a target sequence in a mixture of DNA without cloning or purification. The process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complimentary sequences within the target molecule. Following annealing, the primers are extended with a polymerase so as to form a new pair of complimentary strands. These steps are repeated many times to obtain a high concentration of an amplified segment of the desired target sequence.

The terms “sexually transmitted disease(s)” or “STD” and “sexually transmitted infection” “STI” are used interchangeably throughout.

According to one embodiment the PCR technology may be real-time PCR (quantitative PCR or qPCR), reverse-transcriptase (RT-PCR), PCR-SSCP (single strand conformation polymorphism), ligase chain reaction (LCR), multiplex PCR or nested PCR.

According to one aspect of the invention, the PCR amplification process may use, but not be limited to, Thermal Cycle Machines. Specific examples of thermal cycle machine which may be used include those made by Roche Diagnostics (6800, 8800 COBAS), Vela Diagnostics (Senotas), Thermo Fischer (Quant Studio Flex 12 K).

The detection of HCV can be of a single type, e.g. HCV 1 or may be of two or more types, such as HCV 2 or 3. In some cases, the detection is of high-risk types.

In some embodiments, the detection of HCV infection is in cisgender patients. In other embodiments, the detection is transgender or non-binary patients.

In some embodiments, Cobas® HIV-1/HIV-2 Qualitative is used on the Cobas® 6800/8800 Systems for urine analytes. The assay is intended to be used as an aid in diagnosis of HIV-1/HIV-2 infection. Detection of HIV-1 or HIV-2 nucleic acid is indicative of HIV-1 or HIV-2 infection, respectively. The assay may also be used as an aid in the diagnosis of infection with HIV-1 and/or HIV-2 in pediatric subjects and pregnant women.

In some embodiments the PlexPCR® VHS assay to HSV type-1 & -2 (HSV-1 & HSV-2), Varicella zoster virus (VZV) and Treponema pallidum (syphilis) was validated using urine along with software analysis. The sensitivity and specificity for HSV-1 and HSV-2 was 97.7% and 99.7%, respectively, validated using urine analyte. The sensitivity and specificity for syphilis using urine analyte was 100%.

The present disclosure will be described with respect to particular embodiments and with reference to certain drawings, but the present disclosure is not limited thereto but only by the claims. The drawings described are only schematic and are non-limiting.

Reference throughout this specification to “one embodiment” or “an embodiment” means that a particular feature, structure or characteristic described in connection with the embodiment is included in at least one embodiment of the present disclosure. Thus, appearances of the phrases “in some embodiments,” “in one embodiment,” or “in an embodiment” in various places throughout this specification are not necessarily all referring to the same embodiment, but may. Furthermore, the particular features, structures, or characteristics may be combined in any suitable manner, as would be apparent to one of ordinary skill in the art from this disclosure, in one or more embodiments.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described herein. In addition, the materials, methods, and examples are illustrative only and not intended to be limiting.

Other types of amplification tests which may be used with the present invention include, but are not limited to: reverse transcription polymerase chain reaction (RT-PCR); reverse transcription loop-mediated isothermal amplification (RT-LAMP); and isothermal amplification including: nicking endonuclease amplification reaction (NEAR), transcription mediated amplification (TMA), loop-mediated isothermal amplification (LAMP), helicase-dependent amplification (HDA), clustered regularly interspaced short palindromic repeats (CRISPR), and strand displacement amplification (SDA).

The disclosure presented herein is believed to encompass at least one distinct invention with independent utility. While the at least one invention may be disclosed in exemplary forms, the specific embodiments thereof as described and illustrated herein are not to be considered in a limiting sense, as numerous variations are possible. Equivalent changes, compositions and methods may be made within the scope of the present disclosure, achieving substantially similar results. The subject matter of the at least one invention includes all novel and on-obvious combinations and sub-combinations of the various elements, features, functions and/or properties disclosed herein and their equivalents.

### EXAMPLE 1

The results of the internal analytical validation experiments for the Cobas® HCV assay on the Cobas® 6800/8800 Systems for in vitro detection of Hepatitis C Virus in clinician-collected urine specimens. This test detects Hepatitis C virus genotypes 1 to 6. The internal validation includes urine as an additional specimen type for Cobas® 6800/8800 Systems (Cobas® HCV Test) designed to detect Hepatitis C Virus in clinical blood samples. Whole organism (HCV virus) spiked samples were used for accuracy, specificity, and sensitivity. The validation report documents the performance of using urine-based testing with Cobas® HCV assay on the Cobas® 6800 System using samples from cisgender, transgender and non-binary persons.

### Overview and Procedures

### Scientific Basis of Test and Explanation of Assays

The Cobas® HCV assay is based on a fully automated sample preparation, then followed by PCR amplification and detection. The cobas® 6800/8800 Systems are comprised of the sample supply module, the transfer module, the processing module, and the analytic module. Data is automatically managed by the cobas® 6800/8800 software, assigning test results for all samples as target not detected, <LLoQ (lower limit of quantitation), >ULoQ (upper limit of quantitation), or if the value is present within the linear range LLoQ≤x≤ULoQ, then it is marked as HCV RNA detected. Results of all sample tests can be reviewed directly on the system screen, printed as a report, or exported. The addition of proteinase and lysis reagent to the sample allow for extraction and release of nucleic acid from all external controls, added armored RNA-QS molecules and patient samples, which binds to magnetic glass particles originating from the MGP Cassette. Sequential wash buffer steps are performed to remove unbound substances possessing potential to inhibit PCR amplification and detection. Through the usage of highly conserved regions of Hepatitis C Virus, forward and reverse primers were formulated to provide selective amplification of the targeted nucleic acid within patient samples using a universal, previously designed PCR amplification profile containing a specific number of cycles and temperature steps. A thermostable DNA polymerase enzyme was used for both PCR amplification and reverse-transcription. Deoxyuridine triphosphate (dept.) is included within the master mix, which is incorporated into the DNA amplicon. Within the first thermal cycle, the AmpErase enzyme removes potentially-contaminating amplicons from previous PCR runs, and will later be heat-inactivated at temperatures greater than 55° C., allowing newly-formed amplicons to remain. Cobas® HCV master mix is comprised of dual detection probes specific for HCV target sequences and the RNA-QS, labelled with target-specific fluorescent reporter dyes to allow for detection of both HCV and RNA-QS targets in two separate target channels. The fluorescent signal of the probe will be suppressed by a quencher dye if not bound to the target sequence. During the amplification step of PCR, hybridization of the probes to specific single-stranded DNA templates results in probe cleavage nearing the 5′-to-3′ nuclease activity region of the DNA polymerase, further resulting in separation of the reporter and quencher dyes thus creating a fluorescent signal. Increasing amounts of cleaved probes are produced throughout each PCR cycle, and the cumulative signal of the reporter dye increases mutually. Real-time detection and discrimination of PCR products is accomplished by measuring fluorescence of released reporter dyes for HCV viral targets and the RNA-QS.

### Procedure and Protocol

### Sample Preparation

1. Prepare a barcoded 13 mL round-bottom secondary tube for each urine specimen to be tested.
2. With clean gloved hands, vortex each primary urine specimen vial for 10 seconds immediately prior to transfer
3. Uncap the primary vial and transfer at least 1.5 mL but no more than 4.0 mL into the prepared barcoded secondary tube from step 1. Always use caution when transferring specimens from primary containers to secondary tube. Always use a new pipette tip for each specimen. Transfer tube to a rack (or cap the secondary tube if testing will be performed at a future time).
4. Re-cap the primary vial with a replacement cap before moving to the next specimen. Store the primary vial upright.
5. Load the racks of uncapped secondary tubes into the Sample Supply Module and process on the Cobas® 6800/8800 Systems for HCV testing.

RT-PCR with Cobas® 6800
1. Log onto the system and press “Start” to prepare the system. Then press “Order Tests” and choose “PreservCyt” for ordering specimens collected in PreservCyt® Solution (this specimen type is chosen regardless of using urine as the specimen type).
2. Refill reagents and consumables as prompted by the system: (1) Load test specific reagent cassette (2) Load control cassettes (3) Load pipette tips (4) Load processing plates (5) Load MGP Reagent (6) Load amplification plates (7) Refill Specimen Diluent (8) Refill Lysis Reagent (9) Refill Wash Reagent.
3. Load specimens onto the system for each primary urine specimen vial as follows: (1) Vortex for 10 seconds (2) Aliquot a minimum of 1 mL of urine specimen into a 13 mL round-bottom secondary tube (3) Transfer tube to rack (4) Load sample rack and clotted tip racks into the sample supply module (5) Confirm samples have been accepted into the transfer module.
4. Start run.

### Results and Clean Up

1. Review and export results
2. Remove sample tubes. If needed, cap any sample tubes meeting the minimum volume requirements for future use.
3. Clean up instrument: (1) Unload empty control cassettes (2) Empty amplification plate drawer (3) Empty liquid waste (4) Empty solid waste

### Result Interpretation

The result interpretation should be as follows:

A valid batch may include both valid and invalid sample results.

The entire run is considered invalid if the negative control, HCV low positive control, or HCV high positive control are invalid. The entire run would then be repeated, including control and sample preparation, reverse transcription, amplification, and detection.

Sample results for Cobas® HCV and are marked with “Yes” or “No” in the “Valid” column, and the “Test Result” column is marked with “Titer” for HCV positive results, or “Target Not Detected” for HCV negative results. A titer below 4.3E+01 IU/mL indicates that HCV RNA is present but not quantifiable, and is thus marked as “<Titer Min”.

The “Target 1 result” column indicates the HCV titer measured for positive HCV results, or will be marked “Target Not Detected” for negative HCV results.

For invalid target results, the original specimen should be re-tested no more than two times to obtain valid results. If the results are still invalid a new specimen should be obtained.

### Validation Strategy and Data Summary for HCV Assays

This test was created for a high-throughput laboratory that has the capacity to process hundreds of samples for a predetermined set of targets. The following performance traits were examined to verify this qualitative test for HCV identification:
1. LOD
2. Accuracy
3. Specificity
4. Reproducibility
5. Summary

A pilot study was used to explore and confirm the LOD of the HCV testing. The LOD of the assay was established by running dilutions (1-1,000 copies/mL) of the HCV whole organism controls (Zeptometrix) on Cobas® 6800. Three replicates of each concentration were processed in one day to gauge the possible LOD of the HCV testing. Based on the Ct cutoff values established by Roche Diagnostics for HCV, the limit of detection was established. For the clinical validation study, 30 positive spiked samples (based on LOD data of 500 copies/mL minimum determined by previous pilot study to determine LOD) and 10 negative samples were tested on Cobas® 6800 to examine sensitivity, specificity, percent agreement and accuracy. The specimen type for evaluation was HCV positive spiked urine samples. For achieving highest levels of confidence there should be no contamination in the negative controls (ENTC and NTC) and the positive controls should match. Experiments were carried out over a period of 3 days by two different technicians. All known spiked positive and negative samples were run in triplicates over three days.

LOD was performed as an analytical validation to provide data representing the range at which specific analytes can be detected based off the input being targeted. In other words, the Ct amplification value should reflect the amount of viral RNA being inputted into the assay. LOD for this analytical validation was evaluated using whole organism HCV (Zeptometrix) at 1 copy/mL input in the Cobas® 6800/8800 Systems for HCV testing. The dilution series was 1,000 copies/mL, 750 copies/mL, 500 copies/mL, 250 copies/mL, 100 copies/mL, 75 copies/mL, 50 copies/mL, 25 copies/mL, 5 copies/mL, 1 copies/mL. Samples were run in triplicates inone day. Ct values for assay replicates were averaged, and the averages were plotted. The LOD was determined between 500-1,000 copies/mL, depending on the uniformity of Ct value of detectable copy number.

According to the data provided, the positive signal began to wear off going lower than 500 copies/mL. Therefore, specimens are required to have at least 500 copies/mL of HCV virus present for detection. It has been concluded that we can deem any Ct value between 20 and 36 to be considered a positive result. However, according to the Cobas® HCV Test for IVD use, a cutoff value of <25 IU/mL was used to determine absence of active HCV infection.

### Clinical Validation Data

The clinical validation study was performed over three days where each spiked sample was run daily to get three readings. A total of 40 positive spiked samples (based on LOD data 500 copies/mL of the determined LOD) and 10 negative samples were tested on Cobas® 6800 to examine the overall performance of the assay for clinical samples. To validate this Cobas® 6800 test for HCV, the following performance characteristics were evaluated:
6. Accuracy
7. Reproducibility
8. Analytical Specificity
9. Specimen Stability
10. Summary of Clinical Validation Data
11. As per laboratory procedures, testing includes inspection of all results.

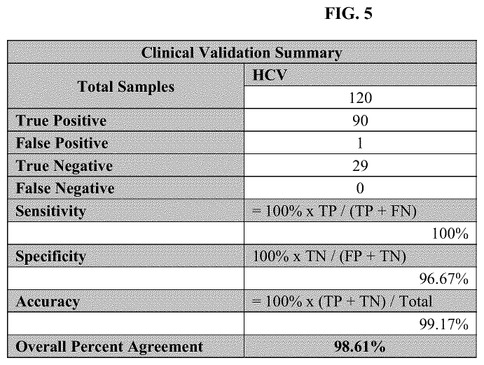

As shown in FIG. 5, the accuracy of controls, for negative controls the expected outcomes should be that all negative controls are all negative.

Therefore, the accuracy should be at 100% for the negative controls. There was one false positive, and all other negative samples resulted negative.

### Negative Control Samples

1. ENTC=ENTC Sample with transport medium only.
2. NTC=NTC Sample with nuclease free water only.

As sown in FIG. 5 for the accuracy of controls, for positive controls all known positive control samples should be positive to reach 100% accuracy.

The acceptable range is 95-100%. The accuracy should be at 100% for the positive controls.

### Stability

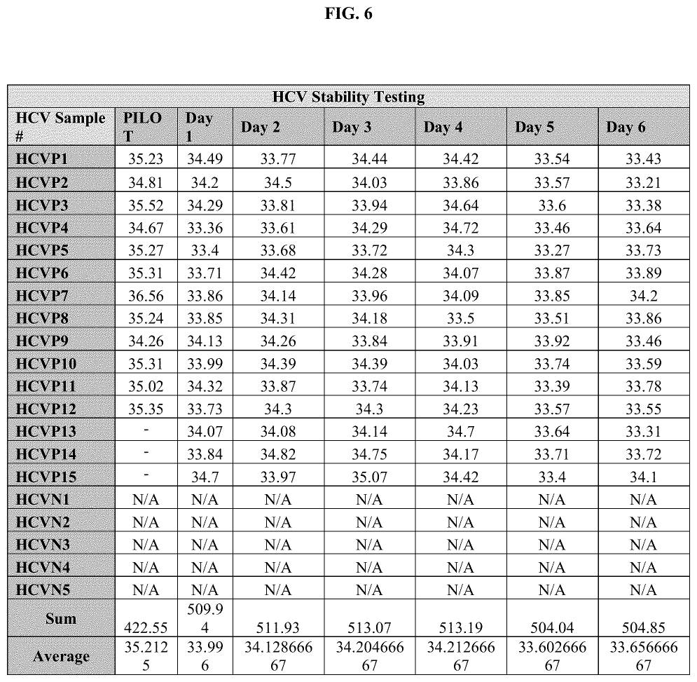

FIG. 6 and FIG. 7 provide stability data. A pilot study of twelve spiked positive HCV samples, and five negative clinical samples was run to determine validity of spiked positive HCV samples after being stored in −80 over the weekend due to laboratory closing. The samples were thawed for four days in 2-8° C., and the stability study ran for the next three days, using samples stored at 2-8° C. for a total of 6 days of testing.

The stability study was designed using a total of twenty clinical urine samples to run for Days 1 through 6 of the study at room temperature (20-27° C.). Fifteen samples were spiked with HCV whole organism control (Zeptometrix) and the remaining five samples were ran as HCV negative clinical samples. All twenty urine samples were run on the Cobas®. The Ct values were recorded and plotted for the complete duration of the stability experiment.

For HCV positive samples, there was a shift of approximately 1 Ct value over the period of 6 days. The urine samples were from cisgender, transgender and non-binary persons.

### EXAMPLE 2

### HIV Validity Test

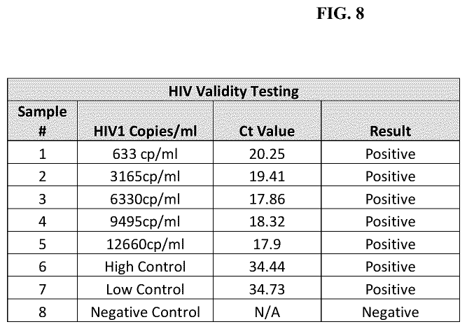

FIG. 8 shows the data for the HIV validity test. The test uses essentially the same PCR procedure as in Example 1 using 8 samples at various dilutions. The modification from previous PCR methods was the use of a self-collected urine specimen from cisgender, transgender and nonbinary persons.

The HIV test is highly reproducible and sensitive using urine analyte. Reproducibility of Cobas® HIV-1/HIV-2 Qualitative was evaluated in urine across reagent lot, test site/instrument system, operator, days, batch, and within batch. Reproducibility testing was performed in three sites using three reagent lots, two Cobas® 6800 Systems and one Cobas® 8800 System, two operators over 6 days; three replicates of each panel member were performed for each batch. Each panel consisted of one negative panel member and six positive panel members. The negative percent agreement was estimated as 100%, with a corresponding 95% exact CI of (98.9%, 100.0%) and the positive percent agreement was 100% for each panel member for both HIV-1 and HIV-2. For HIV-1 positive panel members, the coefficient of variation (CV(%)) for all panel members was ≤1.9%, demonstrating very low variability of Cobas® HIV-1/HIV-2 Qualitative results across reagent lots, sites/instruments, days, operators, and batches.

Overall 1030 urine specimens were tested from subjects known to be HIV-1 positive with HIV-1 viral loads ≥100 copies/mL. There were 736 HIV-1 B subtype specimens and 294 HIV-1 Non-B subtype specimens. The HIV-1 sensitivity of Cobas® HIV-1/HIV-2 Qualitative was 100% (1030/1030, 95% CI: 99.6% to 100%). The sensitivity is for samples with viral RNA concentrations equal to or greater than 100 copies/mL. Similar performance was observed between urine and blood specimens.

The overall HIV-1 specificity and HIV-2 specificity of Cobas® HIV-1/HIV-2 Qualitative was 100%, with no difference between urine and plasma and serum specimen.

### High Throughput HIV-1/HIV-2 Validation Testing

To validate high-throughput HIV-1/HIV-2 laboratory testing that has the capacity to process hundreds of samples for a predetermined set of targets, the following performance traits were examined to verify this qualitative testing:
1. Limit of Detection
2. Accuracy
3. Specificity
4. Reproducibility
5. Summary

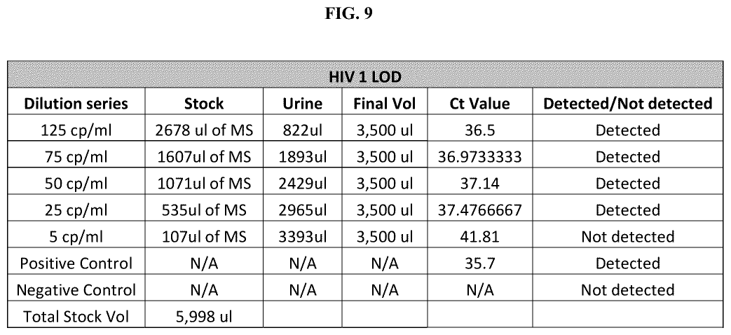

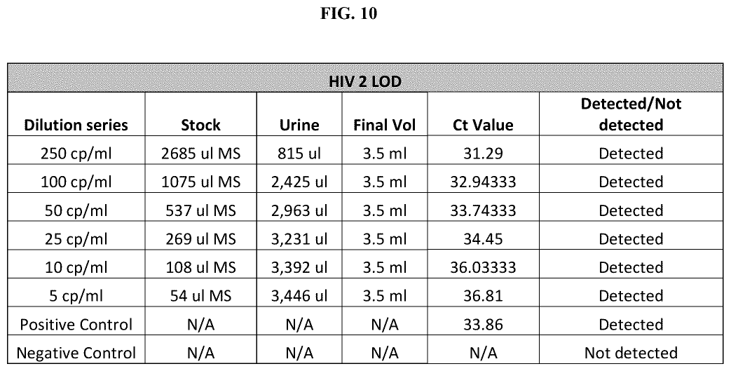

A pilot study that was used to explore and confirm the LOD of the HIV-1/HIV-2 testing. The limit of detection (LOD) of the assay was established by running dilutions (5-125 copies/mL) of the HIV-1 (FIGS. 9) and (5-250 copies/mL) of the HIV-2 (FIG. 10) whole organism controls (Zeptometrix/Exact Diagnostics master stock and Exact Diagnostics) on Cobas® 6800. Three replicates of each concentration were processed in one day to gauge the possible LOD of the HIV-1/HIV-2 testing. Based on the Ct cutoff values established by Roche Diagnostics for HIV-1/HIV-2, the limit of detection was established.

For the clinical validation study, 20 positive spiked samples of HIV-1 (based on LOD data of 25 copies/mL minimum determined by previous pilot study to determine LOD), 20 positive spiked samples of HIV-2 (based on LOD data of 25 copies/mL) and 10 negative samples were tested on Cobas® 6800.

This clinical validation study was tested to examine sensitivity, specificity, percent agreement and accuracy. The specimen type for evaluation was HIV-1and HIV-2 positive spiked urine samples. For achieving highest levels of confidence there should be no contamination in the negative controls (NTC) and the positive controls should match. Experiments were carried out over a period of 3 days. All known spiked positive and negative samples were run in triplicates.

### Limit of Detection (LOD)

Limit of detection (LOD) was performed as an analytical validation to provide data representing the range at which specific analytes can be detected based off the input being targeted. In other words, the Ct amplification value should reflect the amount of viral RNA being inputted into the assay. LOD for this analytical validation was evaluated using whole organism HIV-1 (Zeptometrix/Exact Diagnostics master stock) at 142 copies/mL and whole organism HIV-2 (Exact Diagnostics) at 279.2 copies/mL input in the Cobas® 6800/8800 Systems for HIV-/HIV-2 testing. The dilution series for HIV-1 was 125 copies/mL, 75 copies/mL, 50 copies/mL, 25 copies/mL, 5 copies/mL. The dilution series for HIV-2 was 250 copies/mL, 100 copies/mL, 50 copies/mL, 25 copies/mL, 10 copies/mL 5 copies/mL. Urine samples and stock were run in triplicates in one day. Ct values for assay replicates were averaged, and the averages were plotted. The LOD for HIV-1 was determined between 5-125 copies/mL and the LOD for HIV-2 was determined between 5-250 copies/mL, depending on the uniformity of Ct value of detectable copy number.

### Summary of HIV-1/HIV-2 LOD Results:

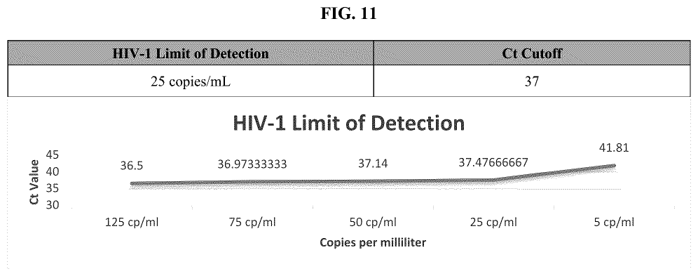

FIG. 11 shows that according to the HIV-1 data obtained, the positive signal began to wear off going lower than 25 copies/mL. Therefore, HIV-1 specimens are preferably required to have at least 25 copies/mL of HIV-1 virus present for detection. It has been concluded from this testing that any Ct value between 30 and 37 is to be considered a positive result.

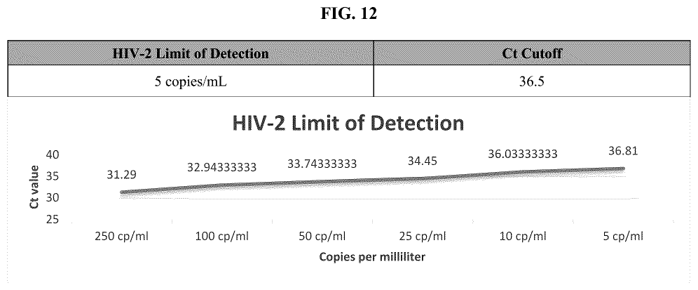

FIG. 12 shows that according to the HIV-2 data provided, the positive signal began to wear off going lower than 5 copies/mill Therefore, HIV-2 specimens are required to have at least 5 copies/mL of HIV-2 virus present for detection. It has been concluded that any Ct value between 30 and 36.5 is to be considered a positive result.

### Clinical Validation Data:

The clinical validation study was performed with a total of 20 positive HIV-1 spiked samples (based on 25 copies/mL of the determined LOD), 20positive HIV-2 spiked samples (based on 5 copies/mL of the determined LOD) and 10 negative samples were tested on Cobas® 6800 to examine the overall performance of the assay for clinical samples. To validate this Cobas® 6800 test for IV-1/HIV-2, the following performance characteristics were evaluated:
1. Accuracy
2. Reproducibility
3. Analytical Specificity
4. Specimen Stability
5. Summary of Clinical Validation Data

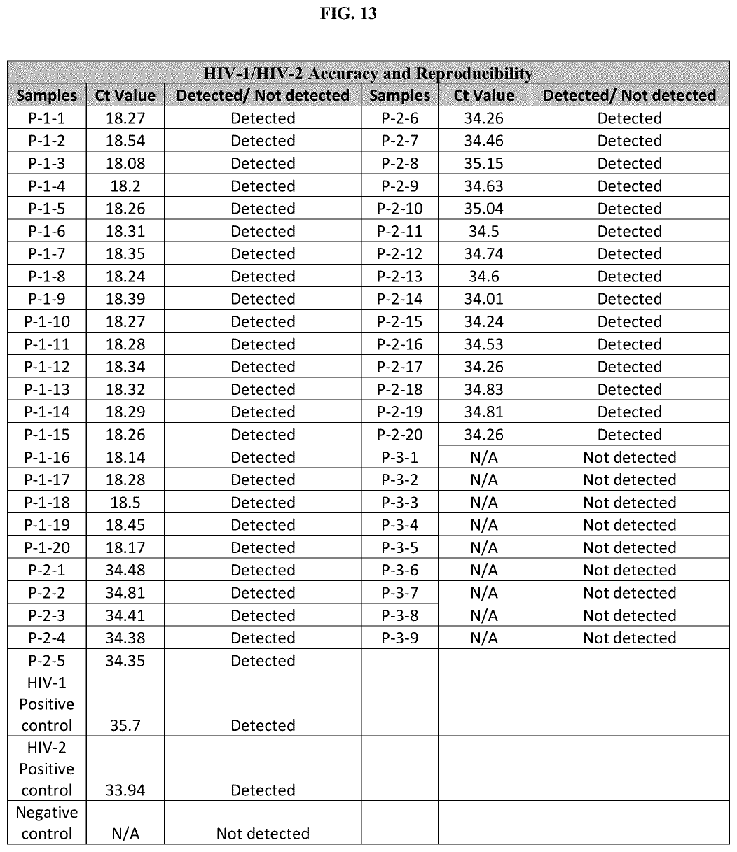

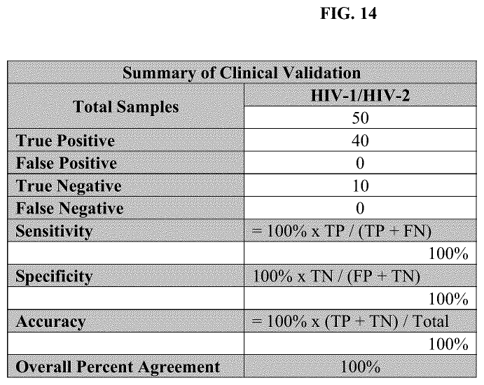

The results of the accuracy and reproducibility testing are shown in FIG. 13. FIG. 14 provides a summary of the Clinical Validation testing.

### EXAMPLE 3

CT/NG and TV/MG Validity Tests

The Cobas CT/NG was validated on the Cobas 6800/8800 software using urine specimens and PCR testing. Cobas® CT/NG can be run with a minimum required sample volume of 1.2 mL for urine specimens. The specimens were from cisgender, transgender and non-binary persons.

The sensitivity and specificity for all tests using urine analyte PCRs tests was between 98-100%.

### EXAMPLE 4

### Herpes and Syphilis Validity Tests

APlexPCR® VHS is a single-well, mulitplex qPCR test detecting common causes of genital and oral lesions. This assay can detect Herpes simplex virus type-1 & -2 (HSV-1 & HSV-2), Varicella zoster virus (VZV) and Treponema pallidum (syphilis) along with software analysis. PlexPCR® VHS is powered by PlexPCR® proprietary technologies for improved multiplex performance compared with other probe-based methods.

The test was validated using the Cobas 6600 and 8800 assay and urine analyte. The APlexPCR VHS test was also validated using urine samples. The specimens were from cisgender, transgender and non-binary persons.

The PlexPCR® VHS assay to HSV type-1 & -2 (HSV-1 & HSV-2), Varicella zoster virus (VZV) and Treponema pallidum (syphilis) was validated using urine along with software analysis. The sensitivity and specificity for HSV-1 and HSV-2 was 97.7% and 99.7%, respectively, validated using urine analyte. The sensitivity and specificity for syphilis using urine analyte was 100%.

What is claimed is:

1. A method of testing for the presence of a pathogen in a patient sample comprising:
a) providing a urine specimen from a patient,
b) extracting DNA in the urine specimen to obtain extracted DNA,
c) subjecting the extracted DNA to an amplification process to obtain amplified DNA, and
d) testing the amplified DNA for the presence or absence of the pathogen, wherein steps a-d do not require manual manipulation.

2. The method of claim 1, wherein the pathogen is human immunodeficiency virus (HIV), hepatitis C virus (HCV), herpes simplex virus (HSV), syphilis, Chlamydia trachomatis (CT), Neisseria gonorrhoeae (NG), Mycoplasma genitalium (MG) or Trichomonas vaginalis (TV).

3. A method of testing for the presence of a pathogen in a patient sample comprising:
a) Providing a urine specimen from a patient,
b) Extracting DNA from the urine specimen to obtain extracted DNA,
c) Subjecting the extracted DNA to an amplification process to obtain amplified DNA, and
d) Testing the amplified DNA for the presence or absence of the pathogen, wherein the pathogen is HIV, HCV, HSV, and/or syphilis.

4. The method of claim 1 or 3, wherein the patient is cisgender, transgender or nonbinary.

5. The method of claim 1 or 3, wherein the urine sample is an unaltered urine sample.

6. The method of claim 1 or 3, wherein the DNA is amplified by a polymerase chain reaction (PCR).

7. The method of claim 1 or 3, wherein the method tests for the presence of at least one pathogen in the specimen.

8. The method of claim 6, wherein the method tests for the presence of multiple pathogens.

9. The method of claim 1 or 3, wherein at least one of the extracting, amplifying and testing is performed by an automated system.

10. The method of claim 9, wherein the system is a Cobas® system.

11. The method of claim 1 or 3, wherein the method further comprises determining whether the patient is positive for the pathogen.

12. The method of claim 9, wherein the method further comprises generating a report indicating whether the patient is positive for the pathogen.

13. The method of claim 1 or 3, where in at least one step of the method can be performed at home.

14. The method of claim 1 or 3, wherein the DNA is extracellular DNA or intracellular DNA.

15. The method of claim 14, wherein the DNA is from the pathogen.
